(19) United States
(12) Patent Application Publication
(10) Pub. No.: US 2026/0021289 A1
(43) Pub. Date: Jan. 22, 2026

(54) BLOOD PUMP HAVING A CATHETER WITH VARYING OUTER DIAMETER AND METHOD OF USE

(71) Applicant: ABIOMED, Inc., Danvers, MA (US)

(72) Inventors: Brian Chouinard, Danvers, MA (US); David Filibiak, Danvers, MA (US)

(73) Assignee: ABIOMED, Inc., Danvers, MA (US)

(21) Appl. No.: 19/272,452

(22) Filed: Jul. 17, 2025

Related U.S. Application Data
(60) Provisional application No. 63/672,919, filed on Jul. 18, 2024.

(51) Int. Cl. A61M 60/13; A61M 60/216; A61M 60/295; A61M 60/31; A61M 60/38; A61M 60/411; A61M 60/497; A61M 60/865

(52) U.S. Cl. CPC A61M 60/13; A61M 60/216; A61M 60/295; A61M 60/31; A61M 60/38; A61M 60/411; A61M 60/497; A61M 60/865

(57) ABSTRACT

Provided herein is a catheter having a varying outer diameter to be used with a percutaneous blood pump and method of use. The system may comprise a catheter having at least two portions, each portion having a different outer diameter. The system may further comprise an introducer sheath. The portion of the catheter having the smaller outer diameter may pass through the introducer sheath. Further, a plurality of catheters having varying outer diameters may pass through a singular introducer sheath. The method may comprise advancing a catheter until the portion having the smaller outer diameter is through the introducer sheath and then inserting a second catheter through the same introducer sheath.

### CROSS-REFERENCE TO RELATED APPLICATIONS

The present application claims priority to U.S. Provisional Patent Application No. 63/672,919, filed Jul. 18, 2024, the contents of which are incorporated by reference herein in its entirety.

### TECHNICAL FIELD

Embodiments disclosed herein relate to medical devices, such as catheters and catheter-based devices.

### BACKGROUND

Currently, percutaneous mechanical support devices are leveraged for a variety of clinical indications. Such support devices may comprise, but are not limited to, an Impella® pump, an Extracorporeal Membrane Oxygenation (ECMO) pump, and a balloon pump. The Impella® pump may further comprise an Impella CP® pump, Impella 5.5® pump, Impella RP® pump, and/or an Impella RP Flex® pump, all of which are by Abiomed, Inc. of Danvers, Mass. Most often they are inserted into a patient percutaneously through a single access point (e.g., radial access, femoral access, axillary access) while other procedures, such as, for example, percutaneous coronary intervention (PCI) are performed through a second access point, such as a contralateral femoral or radial access point. The use of multiple devices on a patient at the same time therefore often requires multiple access sites which presents several challenges.

### SUMMARY OF INVENTION

In some embodiments, an intravascular blood pump may be provided. The intravascular blood pump may include an elongated catheter having a proximal portion, a distal portion and an intermediate portion extending between the proximal and distal portions. The intravascular blood pump may also include a blood pump coupled to the distal portion of the catheter. The proximal portion and distal portion have a first outer diameter, and the intermediate portion a second outer diameter smaller than the first outer diameter.

In some embodiments, an intravascular blood pump may be provided. The intravascular blood pump may include an elongated catheter having a first portion and a second portion extending proximally from the first portion. The intravascular blood pump may include a blood pump coupled to a distal end of the first portion. The first portion may have a first outer diameter and the second portion may have a second outer diameter smaller than the first outer diameter.

In some embodiments, a method may be provided. The method may include positioning at least a portion of an introducer sheath in a patient's vasculature. The method may include inserting a blood pump into a patient's vasculature through the introducer sheath, the blood pump connected to a catheter having a larger diameter portion and a smaller diameter portion, the smaller diameter portion positioned proximally to the larger diameter portion. The method may include advancing the catheter through the introducer sheath such that only a portion of the smaller diameter portion extends through the introducer sheath. The method may include inserting a second device into the patient's vasculature through the introducer sheath while only the smaller diameter portion is within the sheath.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1 shows a patient's heart with a blood pump inserted into the left ventricle;

FIG. 2 shows a patient's heart with a blood pump inserted into the right ventricle;

FIG. 3 shows an illustrative side view of a catheter;

FIG. 4A shows an illustrative side view of a catheter;

FIG. 4B shows an illustrative side view of a catheter;

FIG. 5A shows an illustrative side view of a catheter;

FIG. 5B shows an illustrative side view of a catheter;

FIG. 6 shows an illustrative cross section of an exemplary embodiment, according to aspects of the disclosure;

FIG. 7 shows an illustrative cross section of an exemplary embodiment, according to aspects of the disclosure; and

FIG. 8 illustrates a method according to one embodiment.

### DETAILED DESCRIPTION

As described herein, use of multiple devices on a patient at the same time necessitates more than one access point, but this can be challenging for a variety of reasons. Firstly, the patient may not have two anatomically available access sites for the PCI procedural devices, in which, for example, two 6-7 French sheaths may be used to facilitate procedures such as ballooning and stenting. In addition, peripheral artery disease, vessel lumen size (too small), scar tissue from previous procedures, and other diseases may complicate gaining access to a percutaneous site for larger devices, e.g., mechanical support devices. Using multiple access sites may also increase the likelihood of encountering vascular access complications, which can correlate to increased mortality, added hospital costs, etc. Further, multiple access sites require more procedural time since access needs to be gained more than once, and can lead to increased procedural costs due to requiring multiple vascular closure devices, additional introducers, etc. There is thus a significant need for reducing the complexity of procedures requiring the operation of multiple devices on a patient. Thus, single-access is a valuable technique that allows multiple devices to pass through a single access site. However, the amount of available space in a single access site is limited. Accordingly, the inventors have recognized the value in decreasing the outer diameter of at least the portion of the catheter that crosses the access site to decrease the space consumed by the catheter. The inventors have also recognized the benefit of a larger outer diameter at the distal end of the catheter to provide more rigid support, thus increasing the case of insertion and/or retraction of the catheter and/or blood pump. In some embodiments, a blood pump with a catheter having at least two portions, each with different diameters, is described. At least one of the portions may have a smaller diameter than the other portion(s). The portion with the smaller diameter may pass through an introducer sheath. In other embodiments, there may be more than one catheter passing through the introducer sheath.

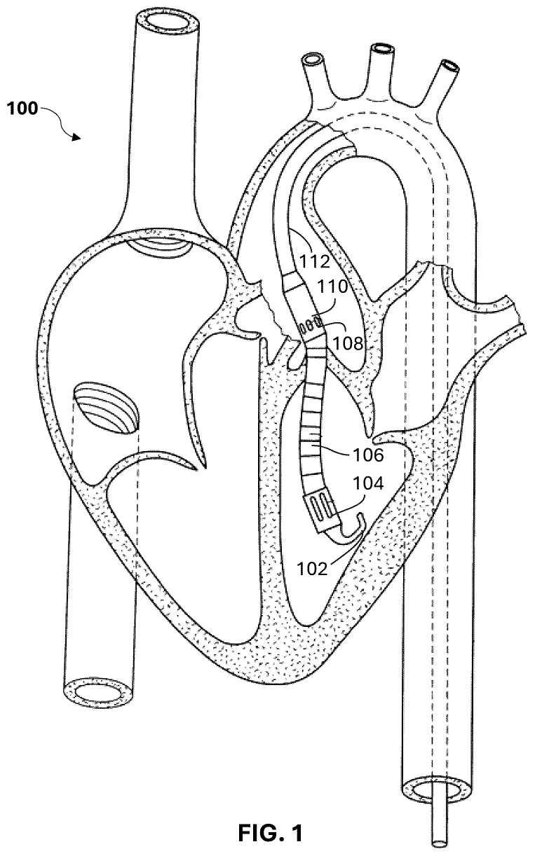

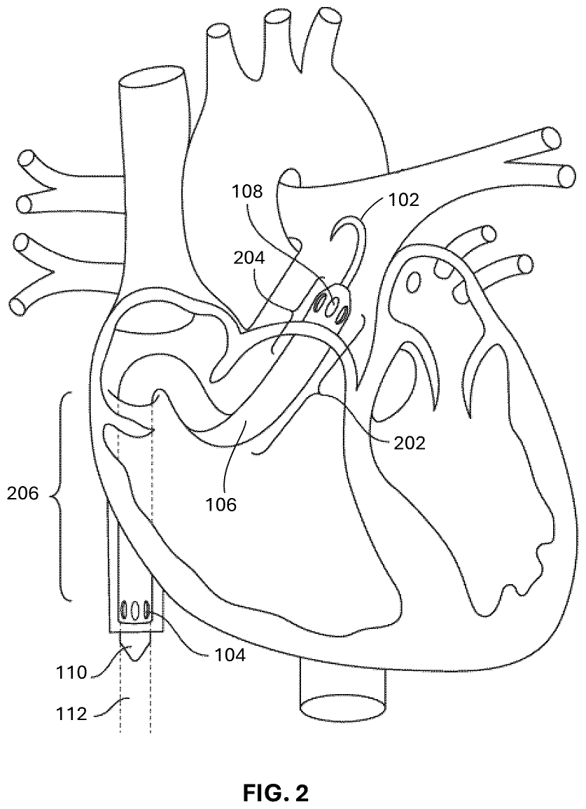

Turning to the figures, FIG. 1 shows a view of a blood pump system, such as an intravascular blood pump system, according to one embodiment of the present disclosure. The blood pump system 100 may include an atraumatic tip 102, a blood flow inlet 104, a cannula 106, a blood flow outlet 108, a blood pump 110, and a catheter 112. In some embodiments, the blood pump 110 may include a pump portion (not shown) and a motor portion (not shown). In such embodiments, the motor portion may be attached to the pump portion. In other embodiments, the motor may be located outside of the patient (extracorporeally). In some embodiments, the blood pump system 100 may be positioned in the left ventricle of a patient, as illustrated in FIG. 1. In some embodiments, the cannula 106 may be expandable. In some embodiments, the blood pump system 100 may not include an atraumatic tip 102. In other embodiments, the blood pump system 100 may be positioned in the right ventricle of a patient, as illustrated in FIG. 2. In the embodiment illustrated in FIG. 2, the blood flow inlet 104 may be positioned in the inferior vena cava, along with some or all of a first end 206 of the cannula 106, and the blood flow outlet 108 may be positioned across the pulmonary valve, along with some or all of a second end 202 of the cannula 106. In some embodiments, the blood pump system 100 shown in FIG. 2 may not include an atraumatic tip 102. In some embodiments, the blood pump system 100 shown in FIG. 2 may have a cannula 106 that is expandable.

In some embodiments, the blood pump system 100 shown in FIG. 1 may be adapted for left heart support, in accordance with some aspects of the disclosure. In such embodiments, the blood pump system 100 may be inserted percutaneously. For example, when used for left heart support, the blood pump system 100 may inserted via a catheterization procedure through the femoral artery or axillary artery, into the aorta, across the aortic valve, and into the left ventricle. Once positioned in this way, the blood pump system 100 may deliver blood from the blood flow inlet 104, which sits inside the left ventricle, through the cannula 106, to the blood flow outlet 108, which sits inside the ascending aorta.

In some embodiments, the blood pump system 100 shown in FIG. 2 may be adapted for right heart support, in accordance with some aspects of the disclosure. In such embodiments, the blood pump system 100 may also be inserted percutaneously. For example, when used for right heart support, the blood pump system 100 may inserted via a catheterization procedure through the femoral vein, into the inferior vena cava, through the right atrium, across the tricuspid valve, into the right ventricle, through the pulmonary valve, and into the pulmonary artery. Once positioned in this way, the blood pump system 100 may deliver blood from the blood flow inlet 104, which sits inside the inferior vena cava, through the cannula 106, to the blood flow outlet 108, which sits inside the pulmonary artery. In some embodiments, the blood pump system 100 may be inserted via the internal jugular (IJ) vein. For example, in such embodiments, the blood pump system 100 may be inserted via a catheterization procedure through the IJ vein, into the superior vena cava, through the right atrium, across the pulmonary valve, and into the pulmonary artery. Once positioned in this way, the blood pump system 100 may deliver blood from the blood flow inlet 104, which sits inside the superior vena cava, through the cannula 106, to the blood flow outlet, which sits inside the pulmonary artery. Here, a portion 204 of the blood pump system may extend across a pulmonary valve.

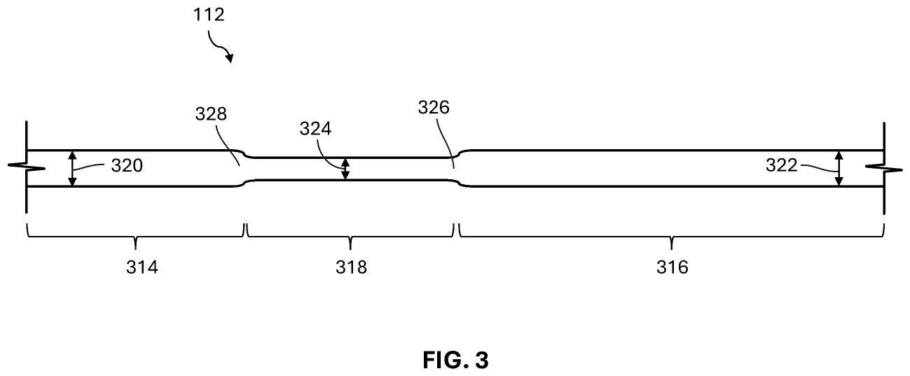

In FIG. 3, the catheter 112 (e.g., an elongated catheter) of the blood pump system 100 is shown. The blood pump system 100 may be coupled to the distal end of the catheter 112, as illustrated in FIG. 1 and FIG. 2. As used herein, distal is understood to mean the direction towards the heart of the patient and proximal is understood to mean the direction away from the heart of the patient. The catheter may have a proximal portion 314, a distal portion 316, and an intermediate portion 318. The intermediate portion 318 may extend distally from the proximal portion 314. The distal portion 316 may extend distally from the intermediate portion 318. The blood pump system 100 may extend distally from the distal end of the distal portion 316. Accordingly, each section of the catheter 112 may have an outer diameter. Each section may have a first outer diameter 320, a second outer diameter 322, and a third outer diameter 324 corresponding to the proximal portion 314, distal portion 316, and the intermediate portion 318, respectively. While diameter is used, there is no requirement that the catheter 112 have a circular cross-section. In some embodiments, diameter may refer to the largest cross-sectional measurement. For example, in some embodiments, the catheter 112 may be an oval shape and thus the diameter may refer to the largest cross-sectional measurement. As will be appreciated, the catheter 112 may be of any suitable cross-sectional geometry.

The first transition region 326 may be located between the distal portion 316 and the intermediate portion 318. The outer diameter of the catheter 112 may increase in the distal direction from the third outer diameter 324 to the second outer diameter 322. In some embodiments, the third outer diameter 324 and the second outer diameter 322 may be the same, or may be substantially equal. Having “substantially equal” diameters is intended to refer to diameters that are ±10%, preferably ±5%, more preferably ±3%, even more preferably ±2%, or still more preferably ±1%, or more preferably exactly equal in size. In some embodiments, the third outer diameter 324 and the first outer diameter 320 may be the same, or may be substantially equal.

The first transition region 326 may be positioned between about 35 centimeters and about 70 centimeters from the distal end of the catheter 112. The first transition region 326 may be positioned between about 45 centimeters and about 60 centimeters from the distal end of the catheter 112. The first transition region 326 may be positioned between about 50 centimeters and about 55 centimeters from the distal end of the catheter 112. As will be appreciated, the first transition region 326 may be positioned at any suitable length from the distal end of the catheter 112.

The second outer diameter 322 may be greater than the first outer diameter 320 and/or the third outer diameter 324 to increase the stability of the catheter 112 and thus aid with insertion and/or retraction of the catheter 112.

In some embodiments, the first outer diameter 320 may be between about 6 French and about 12 French. In some embodiments, the first outer diameter 320 may be between about 7 French and about 11 French. In some embodiments, the first outer diameter 320 may be between about 8 French and about 10 French. In some embodiments, the second outer diameter 322 may also be between about 4 French and about 12 French. In some embodiments, the second outer diameter 322 may also be between about 5 French and about 11 French. In some embodiments, the second outer diameter 322 may also be between about 6 French and about 10 French. In some embodiments, the second outer diameter 322 may also be between about 7 French and about 9 French. In other embodiments, the first outer diameter 320 and the second outer diameter 322 may be the same. In some embodiments, the third outer diameter 324 may be between about 5 French and about 11 French. As will be appreciated, the outer diameters of the catheter 112 may be any suitable size to accommodate different blood pumps in some embodiments. As used herein, a measurement of 1 French is equivalent to ⅓ mm, or 3 mm is equivalent to 1 French.

The second transition region 328 may be located between the intermediate portion 318 and the proximal portion 314. The outer diameter of the catheter 112 may decrease in the distal direction from the first outer diameter 320 to the third outer diameter 324. In some embodiments, the first outer diameter 320 and the second outer diameter 322 may be the same. The second transition region 328 may be positioned between about 85 centimeters and about 120 centimeters from the distal end of the catheter 112. The second transition region 328 may be positioned between about 95 centimeters and about 110 centimeters from the distal end of the catheter 112. The second transition region 328 may be positioned between about 100 centimeters and about 105 centimeters from the distal end of the catheter 112. As will be appreciated, the second transition region 328 may be positioned at any suitable length from the distal end of the catheter 112.

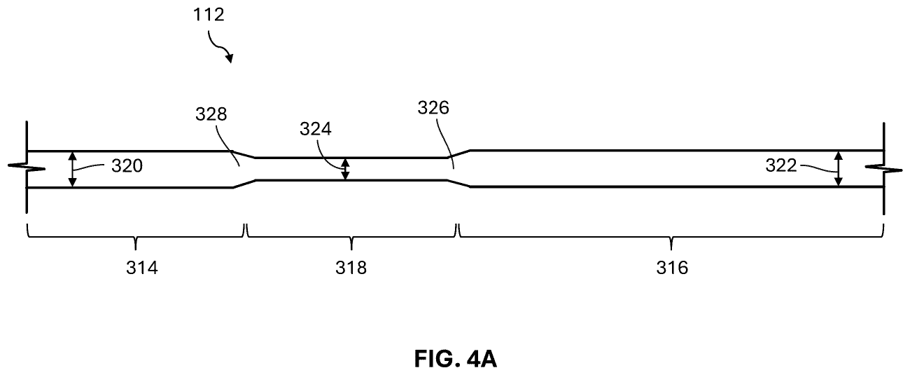

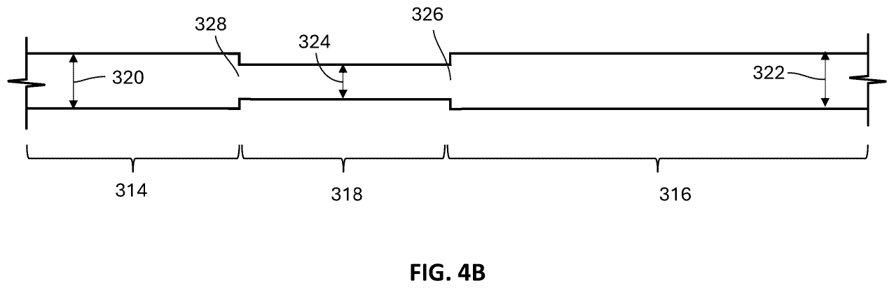

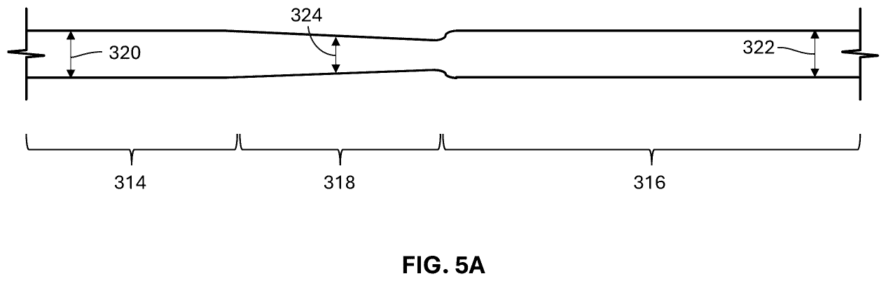

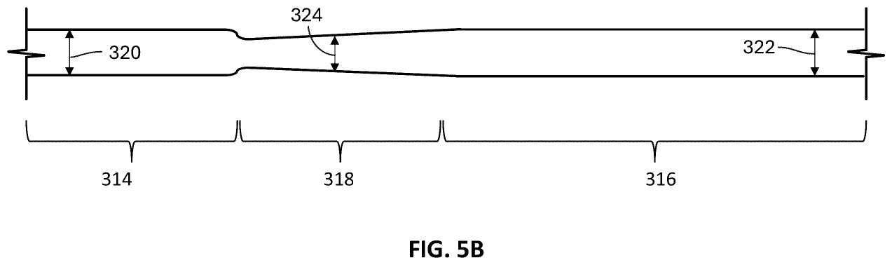

The transition regions 326, 328 may be of different geometries. For example, the outer diameter of the catheter 112 may decrease and/or increase continuously through the transition regions 326, 328, as illustrated in FIG. 3. In another embodiment, the outer diameter of the catheter 112 may decrease and/or increase linearly (as illustrated in FIG. 4A) or in a step-wise manner (as illustrated in FIG. 4B). This may be accomplished by any suitable type of geometry. In some embodiments, the geometry of the transition regions 326, 328 may be different. In some embodiments, the outer diameter of the catheter 112 may decrease continuously through the intermediate portion 318 from the first outer diameter 320 to the second outer diameter 322 as illustrated in FIG. 5A. In other embodiments, the outer diameter of the catheter 112 may increase continuously through the intermediate portion 318 from the third outer diameter 324 to the second outer diameter 322 as illustrated in FIG. 5B. The geometry of the transitions between outer diameters through the intermediate portion 318 may be of any suitable type. In other embodiments, the geometry of transitions between outer diameters may vary along the length of the intermediate portion 318. As will be appreciated, any suitable number of geometries may be used in the transition regions 326, 328.

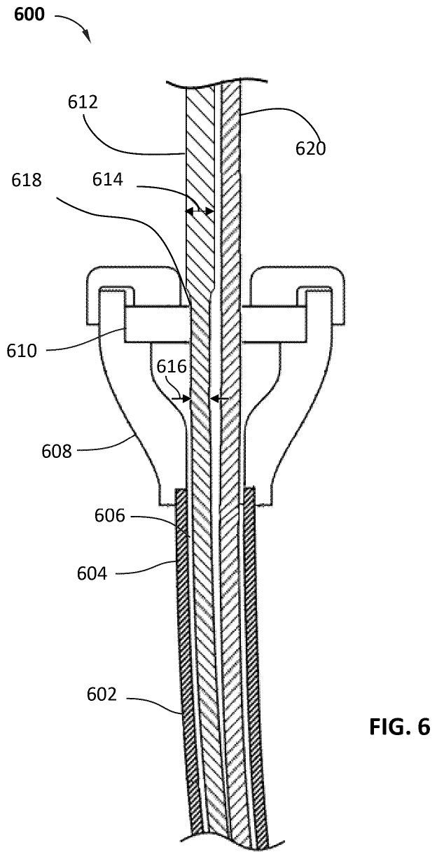

An exemplary embodiment is illustrated in FIG. 6. FIG. 6 shows an introducer sheath delivery system 600 for percutaneously delivering multiple catheter-based medical devices to a patient. System 600 may comprise an introducer sheath 602. The sheath 602 may extend between a proximal end 604 and a distal end (not shown). The sheath 602 may further comprise a lumen 606 that extends between the proximal end 604 and the distal end. System 600 may further comprise a hub 608 coupled to the proximal end 604 of the sheath 602 (sometimes referred to as the sheath body). The hub 608 may comprise a valve 610 to seal the lumen 606 from the ambient. Medical devices, such as catheters, may penetrate the valve 610. In the exemplary embodiment illustrated in FIG. 6, a first catheter 612 may have a varying outer diameter. Portions of the first catheter 612 that are proximal to the valve 610 may have a first outer diameter 614. Portions of the first catheter 612 that are distal to the valve 610 may have a second outer diameter 616. The first outer diameter 614 may be larger than the second outer diameter 616. The first outer diameter 614 may decrease to the second outer diameter 616 in the transition region 618. The transition region 618 may be generally proximal to the valve 610. In some embodiments, a portion of the catheter distal to the valve 610 may have a third outer diameter (not shown). The third outer diameter may be larger than the second outer diameter 616.

In other embodiments, there may be more than one catheter across the same valve 610. For example, as shown in the illustrative embodiment in FIG. 6, a second catheter 620 may cross the same valve as the first catheter 612. In some embodiments, both the first catheter 612 and the second catheter 620 may have varying outer diameters. In some embodiments, only one of the first catheter 612 or the second catheter 620 may have a varying outer diameter. In other embodiments, there may be more than two catheters.

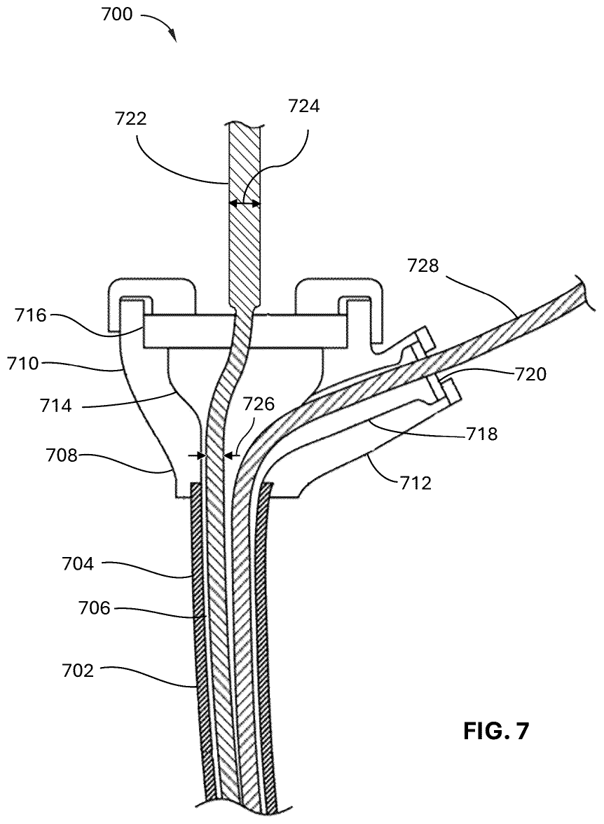

In some embodiments, there may be more than one valve as shown in the exemplary embodiment in FIG. 7. FIG. 7 shows an introducer sheath 702 with a proximal end 704 and a distal end (not shown). The sheath 702 may comprise a lumen 706 extending from the proximal end 704 to the distal end. The sheath 702 may be coupled to a hub 708 at the proximal end 704. The hub 708 may include a first arm 710 and a second arm 712. The first arm 710 may include a first lumen 714 and a first valve 716 coupled to the proximal end of the first lumen 714. The second arm 712 may include a second lumen 718 and a second valve 720 coupled to the proximal end of the second lumen 718. A first catheter 722 may cross the first valve 716. The first catheter 722 may have a varying outer diameter. For example, portions of the first catheter 722 that are proximal to the first valve 716 may have a first outer diameter 724. Portions of the first catheter 722 that are distal to the first valve 716 may have a second outer diameter 726. The first outer diameter 724 may be generally larger than the second outer diameter 726. A second catheter 728 may cross the second valve 720. In some embodiments, the second catheter 728 may have a varying outer diameter. In other embodiments, the second catheter 728 may have a constant outer diameter. In some embodiments, there may be more than two arms. In other embodiments, there may be more than one catheter crossing one or more of the valves.

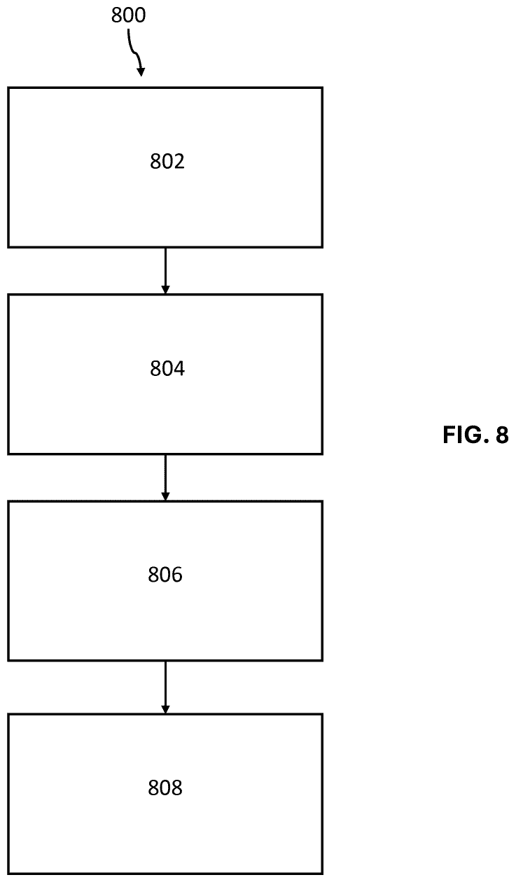

As illustrated in FIG. 8, the method 800 of using more than one catheter-based device across a single-access point according to some aspects of the present disclosure. In some embodiments, the method 800 may include positioning 802 at least a portion of an introducer sheath into a patient's vasculature. In some embodiments, the method may also include inserting 804 a blood pump into a patient's vasculature through the introducer sheath. In such embodiments, the blood pump may be connected to a catheter having a larger diameter portion and a smaller diameter portion. In some embodiments, the smaller diameter portion may be positioned proximally to the larger diameter portion. In some embodiments, the method may also include advancing 806 the catheter through the introducer sheath such that only a portion of the smaller diameter portion extends through the introducer sheath. In some embodiments, the method may also include inserting 808 a second device into the patient's vasculature through the introducer sheath while only the smaller diameter portion is within the sheath. According to some embodiments, the method may include the step of advancing 806 the catheter through the introducer sheath may further include gripping the larger diameter portion during advancement and/or retraction of the blood pump through the introducer sheath.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. An intravascular blood pump comprising:
an elongated catheter having a proximal portion, a distal portion and an intermediate portion extending between the proximal portion and the distal portion; and
a blood pump coupled to the distal portion of the elongated catheter;
wherein the proximal portion and distal portion have a first outer diameter, and the intermediate portion a second outer diameter smaller than the first outer diameter.

2. The intravascular blood pump of claim 1, wherein a first transition from the distal portion to the intermediate portion is positioned between about 35 cm and about 70 cm from a distal end of the elongated catheter.

3. The intravascular blood pump of claim 2, wherein the first transition is positioned between about 45 and about 60 cm.

4. The intravascular blood pump of claim 3, wherein the first transition is positioned between about 50 and about 55 cm.

5. The intravascular blood pump of claim 1, wherein a second transition from the proximal portion to the intermediate portion is positioned between about 85 cm and about 120 cm from a distal end of the elongated catheter.

6. The intravascular blood pump of claim 5, wherein the second transition is positioned between about 95 cm and about 110 cm.

7. The intravascular blood pump of claim 5, wherein the second transition is positioned between about 100 cm and about 105 cm.

8. The intravascular blood pump of claim 1, wherein the blood pump extends distally from the distal portion of the elongated catheter.

9. The intravascular blood pump of claim 1, wherein the blood pump further comprises a motor, the motor attached to the distal portion of the elongated catheter.

10. The intravascular blood pump of claim 1, wherein the blood pump is configured to be inserted into vasculature of a patient through an introducer sheath, the introducer sheath having a hub and a sheath body, wherein at least a portion of the intermediate portion is configured to be in the sheath body when the blood pump is positioned in a heart of the patient.

11. The intravascular blood pump of claim 1, wherein the first outer diameter is between about 6 French and about 12 French.

12. The intravascular blood pump of claim 1, wherein the first outer diameter is between about 7 French and about 11 French.

13. The intravascular blood pump of claim 1, wherein the first outer diameter is between about 8 French and about 10 French.

14. The intravascular blood pump of claim 1, wherein the second outer diameter is between about 5 French and about 11 French.

15. The intravascular blood pump of claim 1, wherein the second outer diameter is between about 6 French and about 10 French.

16. The intravascular blood pump of claim 1, wherein the second outer diameter is between about 7 French and about 9 French.

17. The intravascular blood pump of claim 1, wherein the first outer diameter decreases continuously through a first transition region from the distal portion to the intermediate portion.

18. The intravascular blood pump of claim 1, wherein the first outer diameter decreases in a step-wise fashion through a first transition region from the distal portion to the intermediate portion.

19. An intravascular blood pump comprising:
an elongated catheter having a first portion and a second portion extending proximally from the first portion; and
a blood pump coupled to a distal end of the first portion;
wherein the first portion has a first outer diameter and the second portion has a second outer diameter smaller than the first outer diameter.

20-42. (canceled)

43. A method comprising:
positioning at least a portion of an introducer sheath in a patient's vasculature;
inserting a blood pump into a patient's vasculature through the introducer sheath, the blood pump connected to a catheter having a larger diameter portion and a smaller diameter portion, the smaller diameter portion positioned proximally to the larger diameter portion,
advancing the catheter through the introducer sheath such that only a portion of the smaller diameter portion extends through the introducer sheath; and
inserting a second device into the patient's vasculature through the introducer sheath while only the smaller diameter portion is within the introducer sheath.

44. (canceled)
